(19) United States
(12) Patent Application Publication
(10) Pub. No.: US 2005/0277161 A1
(43) Pub. Date: Dec. 15, 2005

(54) Phosphopeptide antigens associated with MHC molecules

(76) Inventors: Victor H. Engelhard, Crozet, VA (US); Angela Zarling, Charlottesville, VA (US); Donald F. Hunt, Charlottesville, VA (US); Anne M. Evans, Chapel Hill, NC (US); Jeffrey Shabanowitz, Charlottesville, VA (US); Joy M. Polefrone, Charlottesville, VA (US); Leann M. Hopkins, Troy, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/146,854

(22) Filed: Jun. 7, 2005

Related U.S. Application Data
(60) Provisional application No. 60/578,205, filed on Jun. 9, 2004.

(51) Int. Cl. G01N 33/574; G01N 33/00; C07K 14/82

(52) U.S. Cl. 435/7.23; 436/86; 530/352

(57) ABSTRACT

The present invention describes novel tumor-specific phosphorylated peptides, nucleic acids encoding those peptides, and antibodies generated against said peptides. The genes, peptides, and antibodies described herein may be used as diagnostic indicators of the presence of cancer and/or used in therapeutics to treat cancer.

### CROSS REFERENCE TO RELATED APPLICATIONS

This application is entitled to priority pursuant to 35 U.S.C. § 119(e) to U.S. provisional patent application No. 60/578,205 filed Jun. 9, 2004.

### US GOVERNMENT RIGHTS

This invention was made with United States Government support under Grant Nos. RO1 AI20963, RO1 AI133993, and F32 CA09109, awarded by the National Institutes of Health. The United States Government may have certain rights in the invention.

### BACKGROUND

The mammalian immune system has evolved a variety of mechanisms to protect the host from cancerous cells. An important component of this response is mediated by cells referred to as T cells. Cytotoxic T lymphocytes (CTL) are specialized T cells that primarily function by recognizing and killing cancerous cells or infected cells, but they can also function by secreting soluble molecules referred to as cytokines that can mediate a variety of effects on the immune system. T helper cells primarily function by recognizing antigen on specialized antigen presenting cells, and in turn secreting cytokines that activate B cells, T cells, and macrophages.

A variety of evidence suggests that immunotherapy designed to stimulate a tumor-specific CTL response would be effective in controlling cancer. For example, it has been shown that human CTL recognize sarcomas (Slovin et al., 1986, J Immunol 137, 3042-3048), renal cell carcinomas (Schendel et al., 1993, J Immunol 151, 4209-4220), colorectal carcinomas (Jacob et al., 1997, Int J Cancer 71, 325-332), ovarian carcinomas (Peoples et al., 1993, Surgery 114, 227-234), pancreatic carcinomas (Peiper et al., 1997, Eur J Immunol 27, 1115-1123), squamous tumors of the head and neck (Yasumura et al., 1993, Cancer Res 53, 1461-1468), and squamous carcinomas of the lung (Slingluff et al., 1994, Cancer Res 54, 2731-2737; Yoshino et al., 1994, Cancer Res 54, 3387-3390). The largest number of reports of human tumor-reactive CTLs, however, has concerned melanomas (Boon et al., 1994, Annu Rev Immunol 12, 337-365). The ability of tumor-specific CTL to mediate tumor regression, in both human (Parmiani et al., 2002, J Natl Cancer Inst 94, 805-818; Weber, 2002, Cancer Invest 20, 208-221) and animal models, suggests that methods directed at increasing CTL activity would likely have a beneficial effect with respect to tumor treatment.

Melanoma, or skin cancer, is a disease that is diagnosed in approximately 54,200 persons per year. Conventional therapy for the disease includes surgery, radiation therapy, and chemotherapy. In spite of these approaches to treatment, approximately 7,600 individuals die in the United States every year due to melanoma. Overall, the 5-year survival rate for the disease is 88%. The survival rate drops, however, in more advanced stages of the disease with only about 50% of Stage III patients, and 20-30% of Stage IV patients surviving past five years. In patients where the melanoma has metastasized to distant sites, the 5-year survival dips to only 12%. Clearly, there is a population of melanoma patients that is in need of better treatment options. More recently, in an attempt to decrease the number of deaths attributed to melanoma, immunotherapy has been added to the arsenal of treatments used against the disease.

In order for CTL to kill or secrete cytokines in response to a cancer cell, the CTL must first recognize the cancer cell (Townsend and Bodmer, 1989). This process involves the interaction of the T cell receptor, located on the surface of the CTL, with what is generically referred to as an MHC-peptide complex which is located on the surface of the cancerous cell. MHC (major histocompatibility-complex)-encoded molecules have been subdivided into two types, and are referred to as class I and class II MHC-encoded molecules. In the human immune system, MHC molecules are referred to as human leukocyte antigens (HLA). Within the MHC complex, located on chromosome six, are three different loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, and HLA-B8 are examples of different class I MHC molecules that can be expressed from these loci.

The peptides which associate with the MHC molecules can either be derived from proteins made within the cell, in which case they typically associate with class I MHC molecules (Rock and Goldberg, 1999, Annu Rev Immunol 17, 739-779); or they can be derived from proteins which are acquired from outside of the cell, in which case they typically associate with class II MHC molecules (Watts, 1997, Annu Rev Immunol 15, 821-850). The peptides that evoke a cancer-specific CTL response most typically associate with class I MHC molecules. The peptides themselves are typically nine amino acids in length, but can vary from a minimum length of eight amino acids to a maximum of twelve amino acids in length. Tumor antigens may also bind to class II MHC molecules on antigen presenting cells and provoke a T helper cell response. The peptides that bind to class II MHC molecules are generally twelve to nineteen amino acids in length, but can be as short as ten amino acids and as long as thirty amino acids.

The process by which intact proteins are degraded into peptides is referred to as antigen processing. Two major pathways of antigen processing occur within cells (Rock and Goldberg, 1999, Annu Rev Immunol 17, 739-779). One pathway, which is largely restricted to cells that are antigen presenting cells such as dendritic cells, macrophages, and B cells, degrades proteins that are typically phagocytosed or endocytosed into the cell. Peptides derived in this pathway typically bind to class II MHC molecules. A second pathway of antigen processing is present in essentially all cells of the body. This second pathway primarily degrades proteins that are made within the cells, and the peptides derived from this pathway primarily bind to class I MHC molecules. Antigen processing by this latter pathway involves polypeptide synthesis and proteolysis in the cytoplasm, followed by transport of peptides to the plasma membrane for presentation. These peptides, initially being transported into the endoplasmic reticulum of the cell, become associated with newly synthesized class I MHC molecules and the resulting complexes are then transported to the cell surface. Peptides derived from membrane and secreted proteins have also been identified. In some cases these peptides correspond to the signal sequence of the proteins which is cleaved from the protein by the signal peptidase. In other cases, it is thought that some fraction of the membrane and secreted proteins are transported from the endoplasmic reticulum into the cytoplasm where processing subsequently occurs.

Once bound to the class I MHC molecule, the peptides are recognized by antigen-specific receptors on CTL. Several methods have been developed to identify the peptides recognized by CTL, each method of which relies on the ability of a CTL to recognize and kill only those cells expressing the appropriate class I MHC molecule with the peptide bound to it. Mere expression of the class I MHC molecule is insufficient to trigger the CTL to kill the target cell if the antigenic peptide is not bound to the class I MHC molecule. Such peptides can be derived from a non-self source, such as a pathogen (for example, following the infection of a cell by a bacterium or a virus) or from a self-derived protein within a cell, such as a cancerous cell. The tumor antigens from which the peptides are derived can broadly be categorized as differentiation antigens, cancer/testis antigens, mutated gene products, widely expressed proteins, and viral antigens (Castelli et al., 2000, J Cell Physiol 182, 323-331).

Immunization with melanoma-derived, class I or class II MHC-encoded molecule associated peptides, or with a precursor polypeptide or protein that contains the peptide, or with a gene that encodes a polypeptide or protein containing the peptide, are forms of immunotherapy that can be employed in the treatment of melanoma. This form of immunotherapy requires that immunogens be identified so that they can be formulated into an appropriate vaccine. Although a large number of tumor-associated peptide antigens recognized by tumor reactive CTL have been identified, there are few examples of antigens that are derived from proteins that are selectively expressed on a broad array of tumors, as well as associated with cellular proliferation and/or transformation. Attractive candidates for this type of antigen are peptides derived from proteins that are differentially phosphorylated on serine (Ser), threonine (Thr), and tyrosine (Tyr) (Zarling et al., 2000, J Exp Med 192 1755-1762). Due to the increased phosphorylation of cellular proteins in transformed cells as compared to normal cells, there are likely to be new phosphorylated peptides presented on the cell surface available for recognition by CTL. However, these are not predictable from simple inspection of protein sequences, and the exact phosphorylation sites of many proteins, as well as their phosphorylation state in a tumor cell, remain unknown.

There is a long felt need in the art for methods of identifying tumor antigens, and for methods of treating or preventing cancer based on the use of such tumor antigens. The present invention satisfies these needs.

### SUMMARY OF THE INVENTION

The present invention describes novel tumor-specific peptides and antibodies generated against said peptides. The peptides and antibodies described herein may be used as diagnostic indicators of the presence of cancer and/or used in therapeutics to treat and prevent cancer. For the present invention, mass spectrometry has been used to identify phosphorylated peptides associated with the class I MHC molecule HLA-A*0201 and HLA-B7 and displayed on melanoma cells. The invention also provides novel methods for identifying such peptides.

Various aspects and embodiments of the invention are described in further detail below.

### BRIEF DESCRIPTION OF THE DRAWINGS

FIG. 1, comprising FIGS. 1A and 1B, is a graphic illustration of the recognition of naturally processed and presented phosphorylated peptides on cancer cells by the phosphopeptide-specific CTL. Phosphopeptide-specific CTL were incubated with the following cancer cell lines or EBV-transformed B lymphoblastoid cell lines (BLCL): COV413.AAD.A4 ovarian carcinoma, DM331 .AAD.A4 and SLM2.AAD.A1 melanomas, MCF7.AAD.A2 and MDAMB231.AAD breast carcinomas, and JY EBV-BLCL. Supernatants were harvested and evaluated for the presence of murine IFNγ (produced by murine CTL lines). As a positive control, cancer cells were pulsed with the specific phosphopeptide to show that they are capable of presenting exogenously added peptide. In FIG. 1A, two phosphopeptide-specific CTL cell lines, 6850 and 6960 that are specific for the phosphopeptide GLLGpSPVRA, recognize the phosphopeptide on all the cancer cell lines, but not the control cell line. In FIG. 1B, two phosphopeptide-specific CTL cell lines, 5183 and 63 that are specific for the phosphopeptide RVApSPTSGV, recognize the phosphopeptide on all the cancer cell lines, but not the control cell line. The ordinate indicates murine IFNγ in pg/ml. The abscissa indicates each cell line.

### DETAILED DESCRIPTION OF THE INVENTION

### DEFINITIONS

In describing and claiming the invention, the following terminology will be used in accordance with the definitions set forth below.

As used herein, the articles “a” and “an” refer to one or to more than one, i.e., to at least one, of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

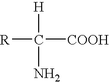

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

A disease or disorder is “alleviated” if the severity of a symptom of the disease or disorder, the frequency with which such a symptom is experienced by a patient, or both, are reduced.

As used herein, the term “antibody” refers to a polyclonal or monoclonal antibody or a binding fragment thereof such as Fab, F(ab′)2 and Fv fragments.

The term “antigen” as used herein is defined as a molecule that provokes an immune response. This immune response may involve either antibody production, or the activation of specific immunologically-competent cells, or both.

As used herein, the term “antigen peptide” refers to a phosphorylated amino acid sequence derived from a cancer cell, such as the sequences selected from the group consisting of SEQ ID NOs:1 through 69.

As used herein, the term “cancer cell-specific phosphopeptide” refers to a phosphopeptide which is expressed at higher levels in said cancer cell compared to its normal counterpart cell or tissue.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control ay be recorded so that the recorded results may be compared with results obtained by examination of a test cell, tissue, sample, or subject.

A “test” cell, tissue, sample, or subject is one being examined.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

As used herein, a “functional” biological molecule is a biological molecule in a form in which it exhibits a property or activity by which it is characterized. A functional enzyme, for example, is one which exhibits the characteristic catalytic activity by which the enzyme is characterized. “Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

By the term “immunizing a subject against an antigen” is meant administering to a subject a composition, a peptide, a polypeptide, or a fragment, derivative, or modification thereof, a protein complex, a DNA encoding a protein complex, an antibody or a DNA encoding an antibody, which elicits an immune response in the human which immune response provides protection to the human against a disease caused by the antigen or an organism which expresses the antigen.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, a “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and Xaa or X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “subject” of diagnosis or treatment is a mammal, including a human.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

By the term “vaccine,” as used herein, is meant a composition, which when inoculated into a mammal has the effect of stimulating a cellular immune response comprising a T cell response or a humoral immune response comprising a B cell response generally resulting in antibody production. The T cell response may be a cytotoxic T cell response directed against macromolecules produced by the bacteria. However, the induction of a T cell response comprising other types of T cells by the vaccine of the invention is also contemplated. A B cell response results in the production of antibody which binds to the antigen. The “vaccine” has the effect of stimulating an immune response in the subject, which serves to fully or partially protect the subject against a disease or its symptoms. The term vaccine encompasses prophylactic as well as therapeutic vaccines. A combination vaccine is one which combines two or more vaccines.

### Embodiments of the Invention

The present invention is directed to novel phosphorylated peptides that give rise to cancer antigens. In one embodiment, for example, the phosphopeptides are those described in Table 1. In one aspect, the peptides bind to MHC molecules. In another aspect, the peptides of the invention stimulate an immune response. In yet another aspect, the peptides of the invention are recognized by a cell or molecule which is a product of, or is stimulated as a result of, an immune response.

TABLE 1
Cancer Antigen Phosphopeptides

| Protein | Gi number | Sequence | HLA type | SEQ ID |
|---|---|---|---|---|
| MUM-2 | 20177848 | RLDpSYVRSL | HLA-A2.1 | 1 |
| Orphan nuclear receptor T2 | 136117 | RQDpSTPGKVFL | HLA-A2.1 | 2 |
| Riken ORF 32, chromosome 10 | 58864795 | VLKGpSRSSEL | HLA-A2.1 | 3 |
| ORF 17, chromosome 2 | 40787650 | RLpSSPLHFV | HLA-A2.1 | 4 |
| ATP-dependent metalloprotease | 14248493 | RLQpSTSERL | HLA-A2.1 | 5 |
| Heterogeneous nuclear ribonucleoprotein: AO | 13938287 | AMAApSPHAV | HLA-A2.1 | 6 |
| Jun-b/c/d | 49456463 | KLApSPELERL | HLA-A2.1 | 7 |
| Ribosomal protein L4 | 22002063 | ILKpSPEIQRA | HLA-A2.1 | 8 |
| Ub-carboxyl terminal hydrolase-10 (USP-10) | 2501458 | KLLpSPSNEKL | HLA-A2.1 | 9 |
| Ribosomal protein S17 | 51476007 | KLLDFGSLpSNLQV | HLA-A2.1 | 10 |
| Krueppel-like zinc finger protein | 903598 | KLLSSAQRpTL | HLA-A2.1 | 11 |
| B-Catenin | 20384898 | YLDpSGIHSGA | HLA-A2.1 | 12 |
| CDC25b:p63 | 14602917 | GLLGpSPVRA | HLA-A2.1 | 13 |
| Insulin receptor substrate-2 | 55663292 | RVApSPTSGV | HLA-A2.1 | 14 |
| Breast cancer anti-estrogen | 55663999 | IMDRpTPEKL | HLA-A2.1 | 15 |
| resistance-3 (BCAR3) | | | | |
| Tumor endothelial marker-6, thyroid | 23451123 | VMIGpSPKKV | HLA-A2.1 | 16 |
| specific PTB domain protein | | | | |
| Hypothetical protein FAM65A protein | 32493393 | RTLpSHISEA | HLA-A2.1 | 17 |
| Nedd4 binding protein 2, BCL3 | 31742492 | KMDpSFLDMQL | HLA-A2.1 | 18 |
| binding protein | | | | |
| Unknown (protein gi: 22902182) | 22902182 | LMFpSVTS(L/I) | HLA-A2.1 | 19 |
| Pleckstrin homology domain-containing | 46397654 | SLQPRSHpSV | HLA-A2.1 | 20 |
| protein family A member 6 | | | | |
| Predicted: similar to RAVER1 | 55648233 | RLLpSPLSSA | HLA-A2.1 | 21 |
| SRp46 splicing factor | 14141201 | SMpTRSPPRV | HLA-A2.1 | 22 |
| Adenosine monophosphate | 56206061 | RQIpSQDVKL | HLA-A2.1 | 23 |
| deaminase 2 (isoform L) | | | | |
| B lymphocyte signal transduction gene | 4261606 | RQApSIELPSM | HLA-A2.1 | 24 |
| B lymphocyte signal transduction gene | 4261606 | RQApSIELPSMAV | HLA-A2.1 | 25 |
| Carcinoembryonic antigen 2b | 3702267 | SLLTFWNL | HLA-A2.1 | 26 |
| SLTP004 | 20146522 | KVQVpTSLSV | HLA-A2.1 | 27 |
| Tsg24 protein | 11967711 | VLLpSPVPEL | HLA-A2.1 | 28 |
| YME1-like 1, isoform 2 | 14043646 | RLQpSTSERL | HLA-A2.1 | 29 |
| FLJ22624 protein | 55661789 | TLApSPSVFKST | HLA-A2.1 | 30 |
| Premature ovarian failure, 1B | 57284143 | RTYpSGPMNKV | HLA-A2.1 | 31 |
| Serine/threonine-protein kinase Chk1. | 7531055 | KLIDIVpSSQKV | HLA-A2.1 | 32 |
| Desmuslin, synemin | 20137613 | RTFpSPTYGL | HLA-A2.1 | 33 |
| FLJ20297 protein, KIAA1418 protein | 40674045 | ALYpSPAQPSL | HLA-A2.1 | 34 |
| Adenosine monophosphate deaminase 2 (isoform L) | 56206061 | RQIpSQDVKL | HLA-A2.1 | 35 |
| BRD4_Human bromodomain containing protein 4 | 20141192 | AVVpSPPALHNA | HLA-A2.1 | 36 |
| MUM-2 | 20177848 | RLDpSYVRS | HLA-A2.1 | 37 |
| Pro-apoptotic protein/BCL2-adenovirus | | HPKRSVpSL | HLA-B7 | 38 |
| E1B interacting protein 3-like/My020 protein | | | | |
| KIAA1187/FLJ10350/Hypothetical protein | | LPApSPRARL | HLA-B7 | 39 |
| FLJ11029 protein/unnamed protein | | HPRpSPTPTL | HLA-B7 | 40 |
| signal-induced proliferation-associated | | YPSpSPRKAL | HLA-B7 | 41 |
| 1 like 1/high-risk human papilloma | | | | |
| viruses E6 oncoproteins targeted protein | | | | |
| Paternally expressed gene 10 ORF1 | | KPRpSPPRAL | HLA-B7 | 42 |
| Novel protein/similar to cdc42 GTPase | | RPAKpSMDSL | HLA-B7 | 43 |
| activating protein | | | | |
| Beta-adrenergic-receptor kinase | | KPRpSPVVEL | HLA-B7 | 44 |
| No direct database hit-(X = L/I) | | RSRpSPRPA(L/I) | HLA-B7 | 45 |
| Suppressor of cytokine signaling protein | | APRpSPPPSRP | HLA-B7 | 46 |
| inositol 1,4,5-triphosphate receptor, type 1 | | RPSGRREpSL | HLA-B7 | 47 |
| Tumor necrosis factor receptor superfamily, | | RPRRpSSTQL | HLA-B7 | 48 |
| member 8 (CD30 antigen) | | | | |
| LIM domain only 6 | | RPRpSPPPRAP | HLA-B7 | 49 |
| General transcription factor 2-I | | RPRpSPGSNSKV | HLA-B7 | 50 |
| Ajuba (a novel LIM protein required for | | GAQPGRHpSV | HLA-B7 | 51 |
| mitotic commitment) | | | | |
| novel retinal pigment epithelial cell | | SPRpSITSTP | HLA-B7 | 52 |
| protein/retinoic acid induced 14 | | | | |
| latent transforming growth factor-beta- | | KARpSPGRAL | HLA-B7 | 53 |
| binding protein-2 | | | | |
| No direct database hit-(X = L/I) | | SPRpSPGRS(L/I) | HLA-B7 | 54 |
| TGFB-induced factor 2/TGIF2 | | LPRGSpSPSVL | HLA-B7 | 55 |
| DNA-directed RNA polymerase I 135 kDa | | FPHpSLLSVI | HLA-B7 | 56 |
| polypeptide/POLR1B protein | | | | |
| thyroid hormone receptor associated protein 3 | | SPRERpSPAL | HLA-B7 | 57 |
| RhoGAP protein/Nadrin | | APRRYpSSSL | HLA-B7 | 58 |
| Synemin/desmuslin | | RTFpSPTYGL | HLA-B7 | 59 |
| numb homolog (Drosophila)-like | | SPFKRQLpSL | HLA-B7 | 60 |
| Chromatin assembly factor 1, subunit A (p150) | | SPRSPpSTTYL | HLA-B7 | 61 |
| No direct database hit-(X = L/I) − MIX? | | RPApSP(K/Q)RA(K/Q)(L/I) | HLA-B7 | 62 |
| Interleukin enhancer-binding factor 3 | 62512150 | KLFPDpTPLAL | HLA-A2.1 | 63 |
| (Nuclear factor of activated T-cells 90 kDa) | | | | |
| Predicted: similar to RAVER1 | 55648233 | RLLpSPLSSA | HLA-A2.1 | 64 |
| MUM-2 (truncated) | 20177848 | RLDpSYVR | HLA-A2.1 | 65 |
| B lymphocyte signal transduction gene | 4261606 | RQApSIELPSM | HLA-A2.1 | 66 |
| KIAA1328 protein -hypothetical protein | 20521886 | KLMpSPKADVKL | HLA-A2.1 | 67 |
| SRp46 splicing factor | 14141201 | SMpTRSPPRV | HLA-A2.1 | 68 |
| TFIID Transcription initiation factor | 5032155 | RLFpSKELRC | HLA-A2.1 | 69 |

In accordance with one embodiment of the present invention, a purified polypeptide is provided comprising the amino acid sequence of SEQ ID NO: 1 through 69, or an amino acid sequence that differs from any of those sequences by one or more conservative amino acid substitutions. In another embodiment the purified polypeptide comprises an amino acid sequence that differs from SEQ ID NO: 1 through 69 by less than 5 conservative amino acid substitutions, and in a further embodiment, by 2 or less conservative amino acid substitutions. In accordance with one embodiment of the present invention a purified polypeptide is provided that consists of the amino acid sequence of SEQ ID NO: 1 through 69, or a bioactive fragment of SEQ ID NO: 1 through 69, or an amino acid sequence that differs from SEQ ID NO: 1 through 69 by one to ten conservative amino acid substitutions. In one aspect, a peptide of the invention is an unphosphorylated peptide having a sequence identical with, or high homologous with, one of the peptides having SEQ ID NOs:1 through 69. The present invention is also directed to isolated nucleic acids which comprise nucleic acid sequences encoding the non-phosphorylated homologs of the phosphopeptides of the invention.

The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid sequence selected from the group consisting of SEQ ID NO: 1 through 69 and a peptide tag, wherein the peptide tag is linked to the phosphorylated peptide sequence. Suitable expression vectors for expressing such fusion proteins and suitable peptide tags are known to those skilled in the art and commercially available. In one embodiment the tag comprises a His tag.

In another embodiment, the present invention is directed to a purified polypeptide that comprises an amino acid fragment of a phosphorylated polypeptide. More particularly the phosphorylated polypeptide fragment consists of natural or synthetic portions of a full-length polypeptide selected from the group consisting of SEQ ID NO: 1 through 69 that are capable of specific binding to their natural ligand. Alternatively, the fragment may comprise an antigenic fragment, including fragments of 10-30, 12-19, 8-12 or 9 amino acids in length, of a polypeptide selected from the group consisting of SEQ ID NO: 1 through 69.

In accordance with one embodiment, a composition is provided for inducing an immune response against a cancer-associated phosphopeptide as described herein. In one embodiment, the composition consists of a peptide comprising a sequence selected from the group consisting of SEQ ID NO: 1 through 69, and antigenic fragments of those sequences. The compositions can be combined with a pharmaceutically acceptable carrier or adjuvant and administered to a mammalian species to induce an immune response. The immune response can take the form of an antibody response, a T helper response, or a CTL response. The immune response may be generated in vitro or in vivo.

In accordance with one embodiment, the peptides can be used to immunize a non-human recipient such as a mouse, rat, or goat for the production of antibodies that specifically recognize the peptides. Antibodies to peptides may be generated using methods that are well known in the art. In one embodiment, recombinantly produced peptides, or fragments thereof are used to generate antibodies against the phosphorylated peptides.

In accordance with one embodiment, an antibody is provided which binds to a polypeptide of the invention. In one aspect, the polypeptide is selected from the group consisting of SEQ ID NOs:1 through 69. In one embodiment the antibody is a monoclonal antibody. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

Antibodies to peptides may be generated using methods that are well known in the art. For the production of antibodies, various host animals, including rabbits, mice, rats, goats and other mammals, can be immunized by injection with a peptide. They may be conjugated to carrier proteins such as KLH or tetanus toxoid. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Methods of immunization to achieve a polyclonal antibody response are well known in the art, as are the methods for generating hybridomas and monoclonal antibodies.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of TAG polypeptides together with genes from a human antibody molecule of appropriate biological activity can be used; such antibodies are within the scope of this invention.

Antibodies generated in accordance with the present invention may include, but are not limited to, polyclonal, monoclonal, chimeric (i.e., “humanized” antibodies), single chain (recombinant), Fab fragments, and fragments produced by a Fab expression library. These antibodies can be used as diagnostic agents for the diagnosis of conditions or diseases characterized by expression or overexpression of antigen peptides (such as cancer), or in assays to monitor a patients responsiveness to an anti-cancer therapy. In one embodiment antibodies specific for one or more of the antigen peptides are used as diagnostics for the detection of the antigen peptides in cancer cells.

The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

In accordance with one embodiment, the detection of antigen peptides is used as a diagnostic mark for detecting cancer. In another embodiment the antigen peptides can be used to immunize an individual to induce an immune response. The induced response may include T helper cells or CTL specific for the antigen peptides. The induced immune response may be useful in preventing the development of cancer in an individual without cancer, and it may be useful in eliminating or preventing the further spread of the disease in an individual with cancer. In one embodiment the antigen peptides may be added to antigen presenting cells. The antigen presenting cells will now present antigen peptides which can be used to stimulate an in vitro T helper cell or CTL response. The T helper cells or CTL can then be used as diagnostics to detect the presence of tumor cells. The T helper cells or CTL can also be infused into a cancer patient as a treatment for cancer.

Accordingly, one embodiment of the invention is directed to the use of antigen peptides as diagnostic markers for neoplastic disease such as cancer. The method comprises the steps of screening for elevated levels or inappropriate expression of antigen peptides, including the expression of antigen peptides in somatic tissues. The term “inappropriate expression” includes any non-typical expression that is deleterious to the cell or host organism, including for example, expression in a cell type that normally does not express the peptide, or expression of a modified form of the peptide that impacts cell function. Such screens could be conducted using antibodies specific for the antigen. Alternatively, antibodies directed against antigen peptides can be used in assays to monitor patients being treated with anticancer therapies to monitor the effectiveness of the therapy.

The antigen peptides are known to be expressed in melanoma, ovarian, breast, colorectal, or squamous carcinoma of the lung, and thus may be used as immunogens to prevent, eliminate, or delay the progression of, inter alia, these types of cancer.

In one aspect, the cancer is, inter alia, sarcoma, renal cell carcinoma, pancreatic carcinomas, squamous tumors of the head and neck

These same antigen peptides may also be expressed in untested forms of cancer and thus may be useful in their ability to prevent, eliminate, or delay the progression of additional cancers.

Antibodies generated with specificity for the antigen peptides are used in accordance with one embodiment to detect the corresponding peptides in biological samples. The biological sample could come from an individual whom is suspected of having cancer and thus detection would serve to diagnose the cancer. Alternatively, the biological sample may come from an individual known to have cancer, and detection of the antigen peptides would serve as an indicator of disease prognosis or treatment efficacy. Appropriate immunoassays are well known in the art and include, but are not limited to, immunohistochemistry, flow cytometry, radioimmunoassay, western blotting, and ELISA. Biological samples suitable for such testing would include, but are not limited to, cells, tissue biopsy specimens, whole blood, plasma, serum, sputum, cerebrospinal fluid, pleural fluid, and urine.

Antigens recognized by T cells, whether helper T lymphocytes or CTL, are not recognized as intact proteins, but rather as small peptides that associate with class I or class II MHC proteins on the surface of cells. During the course of a naturally occurring immune response antigens that are recognized in association with class II MHC molecules on antigen presenting cells are acquired from outside the cell, internalized, and processed into small peptides that associate with the class II MHC molecules. Conversely, the antigens that give rise to proteins that are recognized in association with class I MHC molecules are generally proteins made within the cells, and these antigens are processed and associate with class I MHC molecules. It is now well known that the peptides that associate with a given class I or class II MHC molecule are characterized as having a common binding motif, and the binding motifs for a large number of different class I and II MHC molecules have been determined. It is also well known that synthetic peptides can be made which correspond to the sequence of a given antigen and which contain the binding motif for a given class I or II MHC molecule. These peptides can then be added to appropriate antigen presenting cells, either in vitro or in vivo, and be used to stimulate a T helper cell or CTL response. The binding motifs, methods for synthesizing the peptides, and methods for stimulating a T helper cell or CTL response are all well known and readily available.

The antigens of this invention may take the form of antigen peptides added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigen peptides. The antigen peptides may also be used to vaccinate an individual. The antigen peptides may be injected alone, but most often they would be administered in combination with an adjuvant. The proteins may also be added to dendritic cells in vitro, with the dendritic cells being subsequently transferred into an individual with cancer with the intent of stimulating an immune response.

Peptide analogs can readily be synthesized that retain their ability to stimulate a particular immune response, but which also gain several beneficial features which include, but are not limited to the following: (i) Substitutions may be made in the peptide at residues known to interact with the MHC molecule. Such substitutions can have the effect of increasing the binding affinity of the peptide for the MHC molecule and can also increase the lifespan of the peptide-MHC complex, the consequence of which is that the analog is a more potent stimulator of an immune response than is the original peptide. (ii) The substitutions may be at positions in the peptide that interact with the receptor on the T helper cells or CTL, and have the effect of increasing the affinity of interaction such that a stronger immune response is generated. (iii) Additionally, the substitutions may have no effect on the immunogenicity of the peptide per se, but rather than may prolong its biological half-life or prevent it from undergoing spontaneous substitutions or alternations which might otherwise negatively impact on the immunogenicity of the peptide.

The antigen peptides of this invention can also be used as a vaccine for cancer, and more specifically for melanoma, ovarian, breast, colorectal, or lung squamous cancer. The antigens may take the form of genes, proteins, or peptides. The vaccine may include only the antigens of this invention or they may include other cancer antigens that have been identified. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. The vaccine compositions may further incorporate additional substances to stabilize pH, or to function as adjuvants, wetting agents, or emulsifying agents, which can serve to improve the effectiveness of the vaccine.

The composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time. Alternatively, or concurrently, administration may be by the oral route.

It is understood that the suitable dosage of an immunogen of the present invention will depend upon the age, sex, health, and weight of the recipient, the kind of concurrent treatment, if any, the frequency of treatment, and the nature of the effect desired, however, the most preferred dosage can be tailored to the individual subject, as determined by the researcher or clinician. The total dose required for any given treatment will commonly be determined with respect to a standard reference dose based on the experience of the researcher or clinician, such dose being administered either in a single treatment or in a series of doses, the success of which will depend on the production of a desired immunological result (i.e., successful production of a T helper cell and/or CTL-mediated response to the antigen, which response gives rise to the prevention and/or treatment desired). Thus, the overall administration schedule must be considered in determining the success of a course of treatment and not whether a single dose, given in isolation, would or would not produce the desired immunologically therapeutic result or effect. Thus, the therapeutically effective amount (i.e., that producing the desired T helper cell and/or CTL-mediated response) will depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Needless to say, the efficacy of administering additional doses, and of increasing or decreasing the interval, may be re-evaluated on a continuing basis, in view of the recipient's immunocompetence (for example, the level of T helper cell and/or CTL activity with respect to tumor-associated or tumor-specific antigens).

The concentration of the T helper or CTL stimulatory peptides of the invention in pharmaceutical formulations are subject to wide variation, including anywhere from less than 0.01% by weight to as much as 50% or more. Factors such as volume and viscosity of the resulting composition should also be considered. The solvents, or diluents, used for such compositions include water, possibly PBS (phosphate buffered saline), or saline itself, or other possible carriers or excipients. The immunogens of the present invention may also be contained in artificially created structures such as liposomes, which structures may or may not contain additional molecules, such as proteins or polysaccharides, inserted in the outer membranes of said structures and having the effect of targeting the liposomes to particular areas of the body, or to particular cells within a given organ or tissue. Such targeting molecules may commonly be some type of immunoglobulin. Antibodies may work particularly well for targeting the liposomes to tumor cells.

The present invention is also directed to a vaccine in which a peptide or polypeptide or active fragment of the present invention is delivered or administered in the form of a polynucleotide coding the peptide or polypeptide or active fragment, whereby the peptide or polypeptide or active fragment is produced in vivo. The polynucleotide may be included in a suitable expression vector and combined with a pharmaceutically acceptable carrier.

The vaccine compositions may be used prophylactically for the purposes of preventing cancer in an individual that does not currently have cancer, or they may be used to treat an individual that already has cancer. Prevention relates to a process of prophylaxis in which the individual is immunized prior to the induction or onset of cancer. For example, individuals with a history of severe sunburn and at risk for developing melanoma, might be immunized prior to the onset of the disease. Alternatively, individuals that already have cancer can be immunized with the antigens of the present invention so as to stimulate an immune response that would be reactive against the cancer. A clinically relevant immune response would be one in which the cancer partially or completely regresses and is eliminated from the patient, and it would also include those responses in which the progression of the cancer is blocked without being eliminated.

In one embodiment, the present invention provides methods of screening for agents, small molecules, or proteins that interact with polypeptides comprising a sequence selected from the group consisting of SEQ ID NO: 1 through 69 or bioactive fragments thereof. The invention encompasses both in vivo and in vitro assays to screen small molecules, compounds, recombinant proteins, peptides, nucleic acids, antibodies, etc., which bind to or modulate the activity of antigen peptides and are thus useful as therapeutic or diagnostic markers for cancer. As used herein, modulating the activity of an antigen peptide includes interfering or altering the antigen peptide ligand binding properties.

### EXAMPLES

The invention is now described with reference to the following examples. These examples are provided for the purpose of illustration only and the invention should in no way be construed as being limited to these examples, but rather should be construed to encompass any and all variations which become evident as a result of the teachings provided herein.

### Example 1

Phosphorylated peptides were extracted from melanoma cell lines that express either or both of HLA-B7 and HLA-A*0201, identified by mass spectrometry to be differentially displayed on melanoma versus a control B cell line, and then sequenced. The peptides were identified through the following procedure. Two melanoma cell lines and one B lymphoblastoid cell line were extracted with detergent containing buffer, and HLA-A*0201 class I MHC molecules were purified by immunoaffinity chromatography. Peptides were separated from the MHC molecules by extraction in acid and filtration through a 5000 dalton cut-off filter. Phosphopeptides were identified through analysis by microcapillary reversed phase high performance liquid chromatography tandem mass spectrometry. Sequences were determined from an analysis of collision activated dissociation spectra. Source proteins were determined from a search of protein and DNA databases. SEQ ID NOs:1 through 69 were identified (see Table 1). One of ordinary skill in the art would appreciate that such techniques can be modified and that other techniques are known to aid in identifying peptides of the invention.

These peptides represent potential targets of an immune response, either cytotoxic T lymphocyte or antibody, that could be used for either therapeutic or diagnostic purposes. While the peptides have been identified on melanoma cells, they may also be expressed on other kinds of cancer cells and the invention covers their use for cancers other than melanoma. The binding of these peptides to class I MHC molecules is necessary for their recognition by cytotoxic T lymphocytes, while recognition by antibody could occur in either a class I MHC associated or free form. The invention comprises these peptides, together with structural modifications that retain or enhance the ability: 1) to bind to MHC molecules or; 2 to stimulate an immune response or; 3 to be recognized by a product of an immune response.

The binding of the peptides to class I MHC molecules can be determined by 2-3 anchor residues within the sequence, and these peptides are generally 8-11 residues in length. For example, peptides that bind to HLA-B7 generally contain a Pro at the second position, a hydrophobic aliphatic residue at the carboxyl terminus, and are 9 residues long. However, some peptides have been identified that do not contain one of these anchor residues, and peptides up to 14 residues in length have also been identified. It has also been shown that other residues in a sequence may augment or diminish binding, despite the presence or absence of appropriate anchor residues.

The sequences displayed on a cell are derived from proteolysis of proteins made inside the cell and transported into the lumen of the endoplasmic reticulum. The specificities of the proteases and the transporter are poorly understood, and the sequences of all proteins made by human cells, based on data from the Human Genome Project, are very incomplete. In addition, proteins may undergo modifications such as phosphorylation. However, relatively few of these sites have been identified. Thus, there is a large universe of potential peptides displayed by any given MHC molecule based on the sequences of all proteins made by the cell and the distribution of appropriate anchor motifs within those sequences. However, the exact peptides displayed by a cell are not readily predictable. The direct identification of such peptides by mass spectrometry provides information that cannot be otherwise obtained at present without undue effort.

Different MHC molecules may have similar anchor preferences, leading to the possibility that a peptide associated with, for example, HLA-A*0201, may also be displayed by HLA-A3. The existence of such “supertypes” means that the peptides identified above in association with one MHC molecule may be presented by others, broadening their utility as antigens.

### Example 2

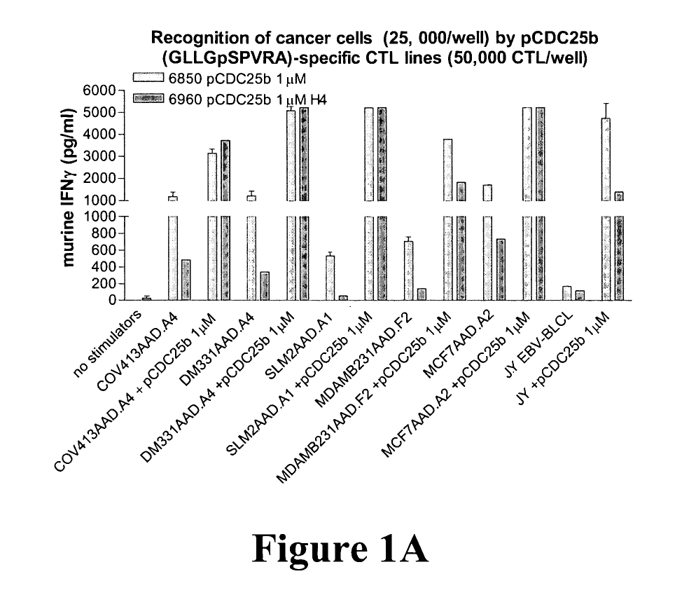

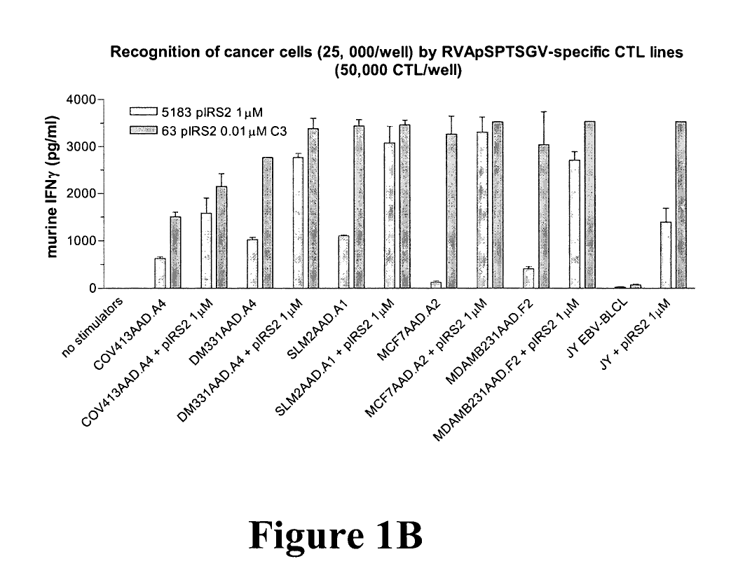

CTL specific for two of the antigen peptides were generated by long term culture with the peptides. Two CTL lines specific for the antigen peptide GLLGpSPVRA, lines 6850 and 6960, SEQ ID NO: 13, and two CTL lines specific for the antigen peptide RVApSPTSGV, SEQ ID NO: 14, lines 5183 and 63 were used to detect these two antigen peptides on cancer cells. The phosphopeptide-specific CTL (50,000 CTL/well) were incubated 24 hours with the following cancer cell lines or EBV-transformed B lymphoblastoid cell lines (BLCL) (25,000 cells/well): COV413.AAD.A4 ovarian carcinoma, DM331.AAD.A4 and SLM2.AAD.A1 melanomas, MCF7.AAD.A2 and MDAMB231.AAD breast carcinomas, and JY EBV-BLCL. Supernatants were harvested 24 hours later and evaluated for the presence of murine IFNγ (produced by murine CTL lines) by ELISA (eBioscience Ready-Set-go murine IFNγ ELISA kit). As a positive control, cancer cells were pulsed with the specific antigen peptide (1 μM) to show that they are capable of presenting exogenously added peptide. In FIG. 1A, two phosphopeptide-specific CTL cell lines, 6850 and 6960, specific for the phosphopeptide GLLGpSPVRA, recognize the phosphopeptide on all the cancer cell lines, but not the control cell line. In FIG. 1B, two phosphopeptide-specific CTL cell lines, 5183 and 63, specific for the phosphopeptide RVApSPTSGV, recognize the phosphopeptide on all the cancer cell lines, but not the control cell line.

The disclosures of each and every patent, patent application, and publication cited herein are hereby incorporated by reference herein in their entirety.

One of skill in the art will appreciate that the superiority of the compositions and methods of the invention relative to the compositions and methods of the prior art are unrelated to the physiological accuracy of the theory explaining the superior results.

Headings are included herein for reference and to aid in locating certain sections. These headings are not intended to limit the scope of the concepts described therein under, and these concepts may have applicability in other sections throughout the entire specification.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. The appended claims are intended to be construed to include all such embodiments and equivalent variations. Accordingly, the present invention is not intended to be limited to the embodiments shown herein but is to be accorded the widest scope consistent with the principles and novel features disclosed herein.

1. A method of identifying a cancer cell-specific phosphopeptide, said method comprising extracting peptides from said cancer cell, identifying phosphopeptides by microcapillary reversed phase high performance liquid chromatography tandem mass spectrometric analysis, and comparing the level of said phosphopeptide in said cancer cell, with the level of said phosphopeptide in an otherwise normal counterpart cell or tissue, wherein a higher level of said phosphopeptide in said cancer cell is an indication that said phosphopeptide is a cancer cell-specific phosphopeptide.

2. The method of claim 1, wherein said phosphopeptide binds to an MHC molecule.

3. The method of claim 1, wherein said phosphopeptide is immunogenic.

4. The method of claim 1, further wherein said extracted peptides are purified by immunoaffinity chromatography, and separated from MHC molecules by acid extraction and filtration through a 5000 dalton cut-off filter, before being identified by microcapillary reversed phase high performance liquid chromatography tandem mass spectrometric analysis.

5. A cancer cell-specific phosphopeptide, and fragments and homologs thereof, identified by the method of claim 1.

6. The cancer cell-specific phosphopeptide of claim 5, wherein said phosphopeptide comprises a sequence selected from the group consisting of SEQ ID NOs:1 through 69, and fragments, homologs, modifications, and derivatives thereof.

7. The method of claim 12, wherein said cancer is selected from the group consisting of melanoma, breast, ovarian, colorectal, and squamous carcinoma of the lung.

8. An antibody that specifically binds to a phosphopeptide of claim 1.

9. The antibody of claim 8, wherein said antibody is a monoclonal antibody.

10. A method for inducing a cytotoxic T lymphocyte (CTL) that is specific for a cancer cell, said method comprising contacting a precursor CTL with an antigenic peptide, wherein said antigenic peptide is a phosphopeptide of claim 1, further wherein said contact occurs under conditions that generate a CTL response to said cancer cell.

11. A method for treating cancer in a subject in need thereof, said method comprising administering to said subject a pharmaceutical composition comprising a therapeutically effective amount of CTLs induced by the method of claim 10.

12. A cancer-specific immunogen, said immunogen comprising a cancer-specific CTL epitope, wherein said epitope comprises a phosphopeptide of claim 1.

13. The cancer-specific immunogen of claim 12, wherein said epitope comprises an amino acid sequence selected from the group consisting of SEQ ID NOs:1 through 69.

14. A method of detecting cancer in a subject, said method comprising:
obtaining a biological sample from a subject;
analyzing the levels of a phosphopeptide of claim 1 in said sample; and
comparing said levels of said phosphopeptide in said subject to the levels of said phosphopeptide in a sample obtained from an otherwise identical control subject, wherein increased levels of said phosphopeptide in said subject compared to the levels of said phosphopeptide in said otherwise identical control subject, indicate the presence of said cancer in said subject.

15. The method of claim 14, wherein said subject is a human.

16. The method of claim 14, wherein said biological sample is selected from the group consisting of cells, tissue biopsy specimens, somatic tissue, whole blood, plasma, serum, sputum, cerebrospinal fluid, feces, pleural fluid, ascites, tears, and urine.

17. The method of claim 14, wherein said phosphopeptide, or a fragment or homolog thereof, is selected from the group of phosphopeptides having an amino acid sequence selected from the group consisting of SEQ ID NOs:1 through 69.

18. A vaccine for treating or preventing a cancer in a subject in need thereof, said vaccine comprising at least one phosphopeptide of claim 1, wherein administration of said vaccine to a subject induces an immune response against said cancer.

19. The vaccine of claim 18, wherein said cancer is selected from the group consisting of melanoma, breast, ovarian, colorectal, and squamous carcinoma of the lung.

20. The vaccine of claim 18, wherein said subject is a human.


SEQUENCE LISTING (69 sequences)

SEQ ID NO: 1 - protein, 9 aa, Homo sapiens
  MOD_RES (4)..(4): PHOSPHORYLATION
RLDSYVRSL

SEQ ID NO: 2 - protein, 11 aa, Homo sapiens
  MOD_RES (4)..(4): PHOSPHORYLATION
RQDSTPGKVFL

SEQ ID NO: 3 - protein, 10 aa, Homo sapiens
  MOD_RES (5)..(5): PHOSPHORYLATION
VLKGSRSSEL

SEQ ID NO: 4 - protein, 9 aa, Homo sapiens
  MOD_RES (3)..(3): PHOSPHORYLATION
RLSSPLHFV

SEQ ID NO: 5 - protein, 9 aa, Homo sapiens
  MOD_RES (4)..(4): PHOSPHORYLATION
RLQSTSERL

SEQ ID NO: 6 - protein, 9 aa, Homo sapiens
  MOD_RES (5)..(5): PHOSPHORYLATION
AMAASPHAV

SEQ ID NO: 7 - protein, 10 aa, Homo sapiens
  MOD_RES (4)..(4): PHOSPHORYLATION
KLASPELERL

SEQ ID NO: 8 - protein, 10 aa, Homo sapiens
  MOD_RES (4)..(4): PHOSPHORYLATION
ILKSPEIQRA

SEQ ID NO: 9 - protein, 10 aa, Homo sapiens
  MOD_RES (4)..(4): PHOSPHORYLATION
KLLSPSNEKL

SEQ ID NO: 10 - protein, 13 aa, Homo sapiens
  MOD_RES (9)..(9): PHOSPHORYLATION
KLLDFGSLSNLQV

SEQ ID NO: 11 - protein, 10 aa, Homo sapiens
  MOD_RES (9)..(9): PHOSPHORYLATION
KLLSSAQRTL

SEQ ID NO: 12 - protein, 10 aa, Homo sapiens
  MOD_RES (4)..(4): PHOSPHORYLATION
YLDSGIHSGA

SEQ ID NO: 13 - protein, 9 aa, Homo sapiens
  MOD_RES (5)..(5): PHOSPHORYLATION
GLLGSPVRA

SEQ ID NO: 14 - protein, 9 aa, Homo sapiens
  MOD_RES (4)..(4): PHOSPHORYLATION
RVASPTSGV

SEQ ID NO: 15 - protein, 9 aa, Homo sapiens
  MOD_RES (5)..(5): PHOSPHORYLATION
IMDRTPEKL

SEQ ID NO: 16 - protein, 9 aa, Homo sapiens
  MOD_RES (5)..(5): PHOSPHORYLATION
VMIGSPKKV

SEQ ID NO: 17 - protein, 9 aa, Homo sapiens
  MOD_RES (4)..(4): PHOSPHORYLATION
RTLSHISEA

SEQ ID NO: 18 - protein, 10 aa, Homo sapiens
  MOD_RES (4)..(4): PHOSPHORYLATION
KMDSFLDMQL

SEQ ID NO: 19 - protein, 8 aa, Homo sapiens
  MOD_RES (4)..(4): PHOSPHORYLATION
LMFSVTSX

SEQ ID NO: 20 - protein, 9 aa, Homo sapiens
  MOD_RES (8)..(8): PHOSPHORYLATION
SLQPRSHSV

SEQ ID NO: 21 - protein, 9 aa, Homo sapiens
  MOD_RES (4)..(4): PHOSPHORYLATION
RLLSPLSSA

SEQ ID NO: 22 - protein, 9 aa, Homo sapiens
  MOD_RES (3)..(3): PHOSPHORYLATION
SMTRSPPRV

SEQ ID NO: 23 - protein, 9 aa, Homo sapiens
  MOD_RES (4)..(4): PHOSPHORYLATION
RQISQDVKL

SEQ ID NO: 24 - protein, 10 aa, Homo sapiens
  MOD_RES (4)..(4): PHOSPHORYLATION
RQASIELPSM

SEQ ID NO: 25 - protein, 12 aa, Homo sapiens
  MOD_RES (4)..(4): PHOSPHORYLATION
RQASIELPSMAV

SEQ ID NO: 26 - protein, 8 aa, Homo sapiens
SLLTFWNL

SEQ ID NO: 27 - protein, 9 aa, Homo sapiens
  MOD_RES (5)..(5): PHOSPHORYLATION
KVQVTSLSV

SEQ ID NO: 28 - protein, 9 aa, Homo sapiens
  MOD_RES (4)..(4): PHOSPHORYLATION
VLLSPVPEL

SEQ ID NO: 29 - protein, 9 aa, Homo sapiens
  MOD_RES (4)..(4): PHOSPHORYLATION
RLQSTSERL

SEQ ID NO: 30 - protein, 11 aa, Homo sapiens
  MOD_RES (4)..(4): PHOSPHORYLATION
TLASPSVFKST

SEQ ID NO: 31 - protein, 10 aa, Homo sapiens
  MOD_RES (4)..(4): PHOSPHORYLATION
RTYSGPMNKV

SEQ ID NO: 32 - protein, 11 aa, Homo sapiens
  MOD_RES (7)..(7): PHOSPHORYLATION
KLIDIVSSQKV

SEQ ID NO: 33 - protein, 9 aa, Homo sapiens
  MOD_RES (4)..(4): PHOSPHORYLATION
RTFSPTYGL

SEQ ID NO: 34 - protein, 10 aa, Homo sapiens
  MOD_RES (4)..(4): PHOSPHORYLATION
ALYSPAQPSL

SEQ ID NO: 35 - protein, 9 aa, Homo sapiens
  MOD_RES (4)..(4): PHOSPHORYLATION
RQISQDVKL

SEQ ID NO: 36 - protein, 11 aa, Homo sapiens
  MOD_RES (4)..(4): PHOSPHORYLATION
AVVSPPALHNA

SEQ ID NO: 37 - protein, 8 aa, Homo sapiens
  MOD_RES (4)..(4): PHOSPHORYLATION
RLDSYVRS

SEQ ID NO: 38 - protein, 8 aa, Homo sapiens
  MOD_RES (7)..(7): PHOSPHORYLATION
HPKRSVSL

SEQ ID NO: 39 - protein, 9 aa, Homo sapiens
  MOD_RES (4)..(4): PHOSPHORYLATION
LPASPRARL

SEQ ID NO: 40 - protein, 9 aa, Homo sapiens
  MOD_RES (4)..(4): PHOSPHORYLATION
HPRSPTPTL

SEQ ID NO: 41 - protein, 9 aa, Homo sapiens
  MOD_RES (4)..(4): PHOSPHORYLATION
YPSSPRKAL

SEQ ID NO: 42 - protein, 9 aa, Homo sapiens
  MOD_RES (4)..(4): PHOSPHORYLATION
KPRSPPRAL

SEQ ID NO: 43 - protein, 9 aa, Homo sapiens
  MOD_RES (5)..(5): PHOSPHORYLATION
RPAKSMDSL

SEQ ID NO: 44 - protein, 9 aa, Homo sapiens
  MOD_RES (4)..(4): PHOSPHORYLATION
KPRSPVVEL

SEQ ID NO: 45 - protein, 9 aa, Homo sapiens
  MOD_RES (4)..(4): PHOSPHORYLATION
RSRSPRPAX

SEQ ID NO: 46 - protein, 10 aa, Homo sapiens
  MOD_RES (4)..(4): PHOSPHORYLATION
APRSPPPSRP

SEQ ID NO: 47 - protein, 9 aa, Homo sapiens
  MOD_RES (8)..(8): PHOSPHORYLATION
RPSGRRESL

SEQ ID NO: 48 - protein, 9 aa, Homo sapiens
  MOD_RES (5)..(5): PHOSPHORYLATION
RPRRSSTQL

SEQ ID NO: 49 - protein, 10 aa, Homo sapiens
  MOD_RES (4)..(4): PHOSPHORYLATION
RPRSPPPRAP

SEQ ID NO: 50 - protein, 11 aa, Homo sapiens
  MOD_RES (4)..(4): PHOSPHORYLATION
RPRSPGSNSKV

SEQ ID NO: 51 - protein, 9 aa, Homo sapiens
  MOD_RES (8)..(8): PHOSPHORYLATION
GAQPGRHSV

SEQ ID NO: 52 - protein, 9 aa, Homo sapiens
  MOD_RES (4)..(4): PHOSPHORYLATION
SPRSITSTP

SEQ ID NO: 53 - protein, 9 aa, Homo sapiens
  MOD_RES (4)..(4): PHOSPHORYLATION
KARSPGRAL

SEQ ID NO: 54 - protein, 9 aa, Homo sapiens
  MOD_RES (4)..(4): PHOSPHORYLATION
SPRSPGRSX

SEQ ID NO: 55 - protein, 10 aa, Homo sapiens
  MOD_RES (6)..(6): PHOSPHORYLATION
LPRGSSPSVL

SEQ ID NO: 56 - protein, 9 aa, Homo sapiens
  MOD_RES (4)..(4): PHOSPHORYLATION
FPHSLLSVI

SEQ ID NO: 57 - protein, 9 aa, Homo sapiens
  MOD_RES (6)..(6): PHOSPHORYLATION
SPRERSPAL

SEQ ID NO: 58 - protein, 9 aa, Homo sapiens
  MOD_RES (6)..(6): PHOSPHORYLATION
APRRYSSSL

SEQ ID NO: 59 - protein, 9 aa, Homo sapiens
  MOD_RES (4)..(4): PHOSPHORYLATION
RTFSPTYGL

SEQ ID NO: 60 - protein, 9 aa, Homo sapiens
  MOD_RES (8)..(8): PHOSPHORYLATION
SPFKRQLSL

SEQ ID NO: 61 - protein, 10 aa, Homo sapiens
  MOD_RES (6)..(6): PHOSPHORYLATION
SPRSPSTTYL

SEQ ID NO: 62 - protein, 10 aa, Homo sapiens
  MOD_RES (4)..(4): PHOSPHORYLATION
RPASPXRAXX

SEQ ID NO: 63 - protein, 10 aa, Homo sapiens
  MOD_RES (6)..(6): PHOSPHORYLATION
KLFPDTPLAL

SEQ ID NO: 64 - protein, 9 aa, Homo sapiens
  MOD_RES (4)..(4): PHOSPHORYLATION
RLLSPLSSA

SEQ ID NO: 65 - protein, 7 aa, Homo sapiens
  MOD_RES (4)..(4): PHOSPHORYLATION
RLDSYVR

SEQ ID NO: 66 - protein, 10 aa, Homo sapiens
  MOD_RES (4)..(4): PHOSPHORYLATION
RQASIELPSM

SEQ ID NO: 67 - protein, 11 aa, Homo sapiens
  MOD_RES (4)..(4): PHOSPHORYLATION
KLMSPKADVKL

SEQ ID NO: 68 - protein, 9 aa, Homo sapiens
  MOD_RES (3)..(3): PHOSPHORYLATION
SMTRSPPRV

SEQ ID NO: 69 - protein, 9 aa, Homo sapiens
  MOD_RES (4)..(4): PHOSPHORYLATION
RLFSKELRC